(19) United States
(12) Patent Application Publication
(10) Pub. No.: US 2016/0030072 A1
(43) Pub. Date: Feb. 4, 2016

(54) GAUGELESS ACTUABLE TIP FOR ATTACHMENT TO HANDPIECE

(71) Applicant: MICROLINE SURGICAL, INC., Beverly, MA (US)

(72) Inventors: Christopher A. DEVLIN, Wakefield, MA (US); Russell LAROCHE, Salem, NH (US); Peter ALISKI, Malden, MA (US); Sharad H. JOSHI, Hopkinton, MA (US)

(21) Appl. No.: 14/450,485

(22) Filed: Aug. 4, 2014

(51) Int. Cl. A61B 17/3201; A61B 17/32; A61B 17/00

(52) U.S. Cl. CPC A61B 17/3201; A61B 17/00234; A61B 17/320016; A61B 2017/00477

(57) ABSTRACT

A removable tip attachable to an instrument handpiece, including a back hub at a proximal end of a tip body and having back hub screw threads configured to threadedly engage outer tube threads of an outer tube of the handpiece, an end effector at a distal end of the tip body, and a yoke at the proximal end of the tip body and having yoke screw threads configured to engage shaft screw threads of an inner shaft of the handpiece, the yoke configured to move within the backhub to actuate the end effector, wherein the yoke screw threads engage with the inner shaft screw threads before the back hub screw threads engage the outer tube threads upon attachment of the tip to the handpiece, and the distance between a proximalmost point of the yoke and a beginning of the back hub screw threads is between 0.533″ and 0.608″.

### BACKGROUND

1. Field of the Invention

This invention relates to a laparoscopic instrument assembly having a removable tip, and in particular, having an actuable removable tip provided with a gaugeless double threaded design.

2. Background of the Invention

Medical procedures such as laparoscopy and the like, which employ a tip at the end of a tube for insertion into the patient, are beneficial because the incisions necessary to perform them are minimal in size, therefore promoting more rapid recovery and lower costs. For example, a patient who undergoes laparoscopic surgery may typically return to normal activity within a period of a few days to about a week, in contrast to more invasive procedures requiring a relatively larger incision (which may require about a month for recovery). (Although the term “laparoscopic” is typically used hereinafter, such use of the term “laparoscopic” should be understood to encompass any such similar or related procedures such as, for example, arthroscopic, endoscopic, pelvoscopic and/or thoroscopic or the like, in which relatively small incisions are used.)

It has been found that in particular medical instruments, such as those employing cutting blades, it is necessary to replace the blades by new and sharper blades in which case it is economically feasible to merely remove the tip of the instrument and replace it with a new tip, rather than discarding the entire instrument.

One such instrument is U.S. Pat. No. 5,358,508, the disclosure of which is expressly incorporated herein by reference in its entirety. As described in FIGS. 1-3, this instrument provides a laparoscopic instrument assembly 10 having a disposable operable tip (also referred to as an end effector) 14, the assembly including an actuator 12 (also referred to as a handpiece) having a tubular sheath 36 with an actuator rod 40 disposed for axial movement within the sheath. A handle 15 is provided for moving the actuator rod 40 axially within the sheath and is disposed adjacent the rear end of the actuator rod, the rod having a continuous internal thread formed at the forward end of the rod opposite the one end, and the sheath structure having a continuous external thread 39 formed thereon adjacent the rod forward end.

The tip 14 includes a pair of cutting blades 20, 21 forming a scissors, the blades being movable to an open and closed position by rotation about a pivot pin 22. The tip 14 further includes a tip rod (also called a yoke) 26 disposed for axial movement within a tubular outer casing structure (also called a backhub) 24 covering an outer tube 38, the tip rod having a plurality of external threads 25 formed on the rear end thereof for mating engagement with the actuator rod's internal threads 42, and the tip casing structure having a plurality of internal threads 34 at the rear end adjacent the one end of the tip rod for mating engagement with the external threads 39 of the sheath 36. The external threads 25 on the yoke 26 engage the internal threads on the actuator rod 40, and the internal threads on the backhub 34 engage the external threads 39 on the outer tube 38.

The actuator 12 is provided with a handle 15 including a pair of thumb and finger grip members 16 and 17 respectively, the finger grip member 17 being stationary with respect to the actuator 12 while the thumb grip member 16 is movable through rotation about a pivot pin 18. A stop 2 on grip 16 sets the position of the blades so that they are completely closed when it engages grip 17 and also establishes the correct position for the threads on the tip to engage the threads on the actuator. The actuator rod 40 has a pin 44 extending therefrom, which is received in an elongated slot 46 formed at the rear end of the outer tube 38 to prevent rotation of the actuator rod 40 within the sheath 36, and to allow movement of the rod in the axial direction.

As surgeons perform laparoscopic or open surgery, the need for a one to one relationship between the moving thumb grip member 16 on the hand piece 12 and the end effector 14 is critical. One situation where this is a major issue is when cutting tissue with scissors 20, 21. Because the scissor is used to nip at tissue, the hysteresis (or play) between the handpiece 16 and the end effector becomes very important. When the surgeon squeezes the thumb grip member 16 of the handpiece, the scissors 20, 21 must move in concert so the surgeon can make the proper cut using the intended motion. If the scissor 20, 21 does not move or if there is hysteresis in the motion, the surgeon may cut tissue unintentionally, possibly resulting in injury to the patient. A situation where the relationship is critical is grasping tissue using opposable graspers (rather than scissors). Because precise pressure is often required to manipulate tissue without causing damage, the one to one relationship and minimized hysteresis of the design is necessary, enhancing the tactile feel of the device.

One way prior art devices maintain the one to one relationship between the moving thumb grip member 16 on the handpiece 12 and the tip 14 is performing a “gauging” operation of the tip 14 during the tip manufacturing process, in which the external threads 25 on the yoke 26 are precisely aligned with the internal threads 34 on the backhub 24 in the circumferential direction. In other words, in the gauging process, the thread starts for each of the threads 24, 25 are always the same circumferentially, which eliminates the possibility of “lock up” during connection of the tip to the handpiece; however, the gauging process is labor-intensive and requires a high degree of precision to make all end effectors 14 fit with all handpieces 12. The reliance on calibrated gauges and high-precision fixturing make this design more costly and susceptible to drift (i.e., an error in the gauging procedure) and inconsistent end effector to hand piece connection. In the event of drift, the end effector must either be re-calibrated or discarded, which is labor intensive and/or costly. There has thus arisen a need for an end effector that is not reliant on a gauging process, while still providing for good one to one feel of the hand piece and end effector.

A ball and clevis design for a handpiece is known in the art; however the required clearances to facilitate ball and clevis assembly create a hysteresis that make the motion of the handle movable member different than the opening and closing of the end effector. Therefore the delicate and close movement requirements of scissors are compromised and can lead to unintentional cutting.

### SUMMARY OF THE INVENTION

A feature of the disclosure provides for gaugeless attachment of a 5 mm coaxially-threaded laparoscopic hand piece and end effector to eliminate lock ups between the hand piece and the end effector without the need to have precise thread-to-thread start relationships between the inner and outer threads on the hand piece and the inner and outer thread of the end effector.

Another feature minimizes, through precision components with tight tolerances, the hysteresis between the handpiece and the actuation of the end effector, to improve a user's tactile feel.

A further feature provides the user with one to one relationship of the handle motion and the end effector motion by using a pin a slot with precise angular alignment.

Yet another feature provides significantly improved clamping force at the fully-closed position of the end effector (particularly when the end effector uses graspers, pincers or similar grabbing tools) by providing overstroke (i.e., when pressure can be increasingly be applied to the tissue even after the end effector is in the fully-closed position) capabilities of the hand piece with respect to the actuation of the end effector using thread pitch differential (between the yoke-rod thread pitch and the backhub-outer sheath thread pitch), thereby providing significantly improved clamping force at the fully-closed position.

The present disclosure adds further features to the inner and outer shafts of the handpiece to prevent the rotation of the inner shaft with respect to the outer shaft to allow for full shaft rotation while maintaining the rotational relationship between the inner and outer shaft of the handpiece.

Further, hysteresis related to any individual handpiece or end effector is minimized by the design and tight tolerances of components and assemblies. However, hysteresis of the “system” (an end effector attached to a handpiece) is overcome through calculated overstroke of end effectors when attached to handpieces.

Also, handpiece hysteresis may be minimized by the use of a ball and socket connection between the inner shaft and thumb loop to provide smooth and precise movement of the inner shaft within the outer shaft. The inner and outer shaft should remain in the same relative position (rotationally) during end effector installation to prevent the threads from locking up and also to insure complete engagement of the end effector to the handpiece. A pin and slot configuration between the inner and outer shafts is used to keep the thread relationship constant between the inner rod thread and the outer shaft thread. Without these features to keep the inner and outer shafts aligned, the inner shaft is subject to rotation independent of the outer shaft resulting in inconsistent and/or incomplete stroke and an adverse effect on tactile feedback.

In another aspect, the thread pitch differential of the dual coaxial thread design and the number of threads that engage when attaching an end effector to a handpiece help provide “overstroke” ability, thereby providing the surgeon with higher grasping forces for graspers and ensuring complete blade crossover (closure) of scissors within the normal stroke of the handpiece.

In a further aspect, the ability to install any end effector on any handpiece without the need for complicated gauging, while insuring integration of the dual coaxial threads. By engaging the inner thread first and allowing the inner shaft of the handpiece to move distally during end effector installation, complicated gauging to accommodate coordinated engagement of both threads simultaneously is eliminated. The distance from thread start to thread start of both handpieces (known as the constrained dimension) and end effectors ensures that:
a. The end effector and the hand piece will not lock up during or after assembly. Any end effector can be fully installed onto any suitable handpiece, ensuring patient and surgeon protection from unintended exposure to electrical energy.
b. Sufficient engagement of both coaxial threads provides adequate overstroke capabilities without impeding full opening of an end effector, to ensure optimal precision and control over the entire intended range of the device.

In accordance with a further aspect, the handpiece and end effector together provide a laparoscopic surgical instrument having an outer diameter of 5 millimeters. In a feature, the hand piece is reusable and the end effector is disposable. The instrument in accordance with a feature uses a coaxial thread based design set to a specific relationship to ensure the thread connection made by the yoke threads is engaged by one thread (i.e., one thread winding) before the backhub thread is engaged.

Accordingly, provided is a removable tip attachable to an instrument handpiece, the tip including a tip body, a back hub at a proximal end of the tip body and having internally-facing back hub screw threads configured to threadedly engage outer externally-facing tube threads of an outer tube of the instrument handpiece, an end effector at a distal end of the tip body, a yoke at the proximal end of the tip body and having externally-facing yoke screw threads configured to threadedly engage internally-facing shaft screw threads of an inner shaft of the instrument handpiece, the yoke configured to slidably and coaxially move within the backhub to actuate the end effector upon sliding actuation of the inner shaft within the outer tube, wherein the yoke screw threads engage with the inner shaft screw threads before the back hub screw threads engage the outer tube threads upon attachment of the tip to the instrument handpiece, and the distance between a proximalmost point of the yoke and a beginning of the back hub screw threads is between 0.533 inches and 0.608 inches.

In another aspect, the distance between a proximalmost point of the yoke and a beginning of the back hub screw threads may be between 0.533 inches and 0.574 inches. Further, the back hub screw threads may be configured to engage the outer tube threads for 3.971 to 4.390 windings after the yoke screw threads engage with the inner shaft screw threads. Also, the back hub screw threads may engage the outer tube threads for 3.971 windings after the yoke screw threads engage with the inner shaft screw threads. Additionally, the backhub may have an outer diameter of 5 millimeters. In a further aspect, the back hub screw threads may be 8-36 screw size, and the yoke screw threads may be 4-32 screw size.

A further aspect may provide a method of removably attaching an instrument tip to an instrument handpiece, the instrument tip having a back hub at a proximal end of a tip body and having internally-facing back hub screw threads, an actuable end effector at a distal end of the tip body, and a yoke coaxially disposed within the tip body, extending from the proximal end of the tip body and having externally-facing yoke screw threads, and the instrument handpiece having an outer tube having externally-facing tube threads, and an inner shaft slidably disposed within the outer tube and having internally-facing shaft screw threads, the method including threadedly engaging the yoke screw threads with the shaft screw threads by at least one winding, and thereafter, threadedly engaging the back hub screw threads with the tube threads by a maximum of 4.1 windings such that the inner shaft distally moves in relation to the outer tube.

In accordance with another aspect, during the threadedly engaging the back hub screw threads with the tube threads, the end effector is not actuated. Also, the inner shaft may include a pin radially extending therefrom, and slidable within an axially extending slot located in the outer tube, the method further including during the threadedly engaging the back hub screw threads with the tube threads, the pin remains out of contact with opposed ends of the slot.

In an additional aspect, the distance between a proximalmost point of the yoke and a beginning of the back hub screw threads may be between 0.533 inches and 0.608 inches, or the distance between a proximalmost point of the yoke and a beginning of the back hub screw threads may be between 0.533 inches and 0.574 inches. Also, the threadedly engaging the back hub screw threads with the tube threads may be for 3.971 windings.

Additionally provided may be an instrument assembly including an instrument tip including a back hub at a proximal end of a tip body and having internally-facing back hub screw threads, an actuable end effector at a distal end of the tip body, and a yoke coaxially disposed within the tip body, extending from the proximal end of the tip body and having externally-facing yoke screw threads, and an instrument handpiece for removably accepting attachment of the tip thereto, including an inner shaft slidably disposed within the outer tube and having internally-facing shaft screw threads threadedly engaging the yoke screw threads, an outer tube having externally-facing tube threads threadedly engaging the back hub screw threads for 3.971 to 4.390 windings after the shaft screw threads engage with the yoke screw threads, such that the tip is affixed to the handpiece.

In an additional aspect, the tube threads may engage the back hub screw threads for 3.971 windings after the shaft screw threads engage with the yoke screw threads. Also provided may be a pin radially extending from the inner shaft, and an axially extending slot located in the outer tube and capturing the pin therewithin, such that the pin remains out of contact with opposed ends of the slot in an unactuated state of the tip.

Also, the distance between a proximalmost point of the yoke and a beginning of the back hub screw threads may be between 0.533 inches and 0.608 inches, or the distance between a proximalmost point of the yoke and a beginning of the back hub screw threads may be between 0.533 inches and 0.574 inches.

Also provided may be an outer sheath covering the outer tube, wherein the backhub and the outer sheath each have an outer diameter of 5 millimeters. In a further aspect, the back hub screw threads and outer tube screw threads may each be a 8-36 screw size, and the yoke screw threads and inner shaft screw may each be a 4-32 screw size.

Other exemplary embodiments and advantages of the present invention may be ascertained by reviewing the present disclosure and the accompanying drawings, and the above description should not be considered to limit the scope of the present invention.

### BRIEF DESCRIPTION OF THE DRAWINGS

The present invention is further described in the detailed description which follows, in reference to the noted plurality of drawings, by way of non-limiting examples of preferred embodiments of the present invention, in which like characters represent like elements throughout the several views of the drawings, and wherein:

FIG. 1 shows a side elevational view showing a laparoscopic instrument assembly constructed in accordance with the related art.

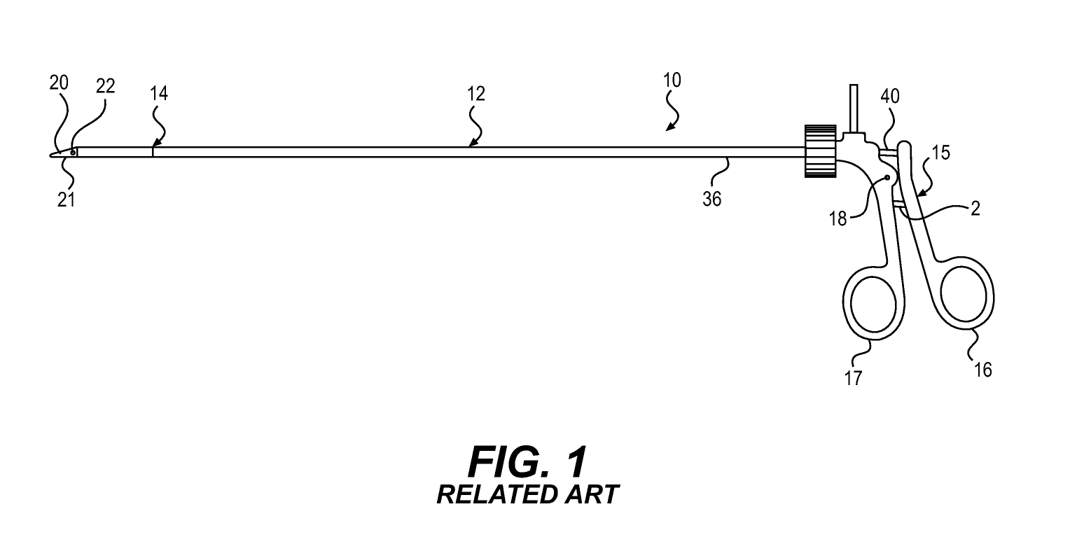

FIG. 2 shows an instrument tip prior to connection to a handpiece in accordance with the related art.

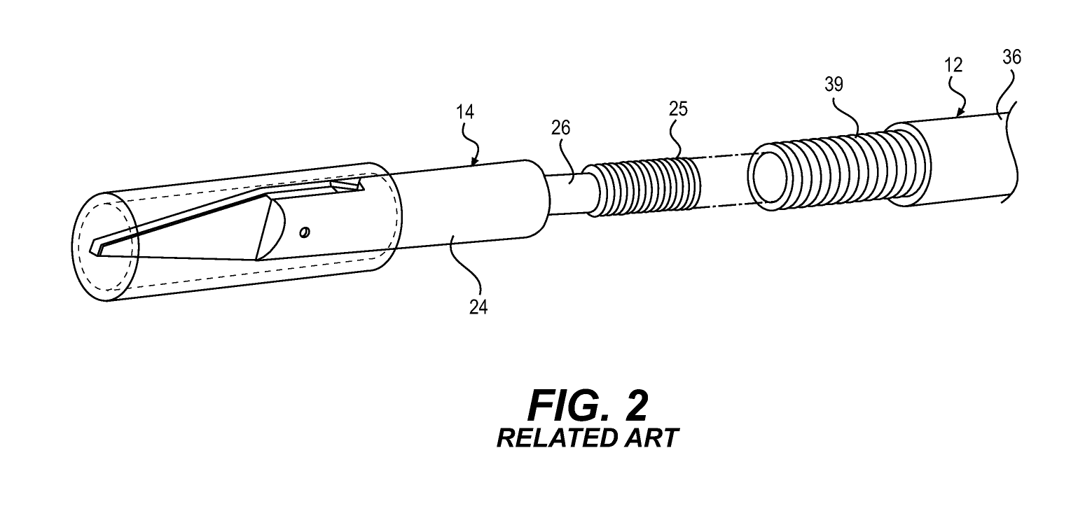

FIG. 3 shows a sectional view of the junction of an instrument tip connected to a handpiece, in accordance with the related art.

FIG. 4 shows a sectional view of an instrument tip and handpiece prior to assembly, in accordance with a feature of the present disclosure.

FIG. 5 shows a side elevational view of the assembled instrument tip and handpiece with the outer sheath removed, in accordance with a feature of the present disclosure.

### DETAILED DESCRIPTION

The particulars shown herein are by way of example and for purposes of illustrative discussion of the embodiments of the present invention only, and are presented for providing what is believed to be the most useful and readily understood description of the principles and conceptual aspects of the present invention. In this regard, no attempt is made to show structural details of the present invention in more detail than is necessary for the fundamental understanding of the present invention, the description taken with the drawings making apparent to those skilled in the art how the several forms of the present invention may be embodied in practice.

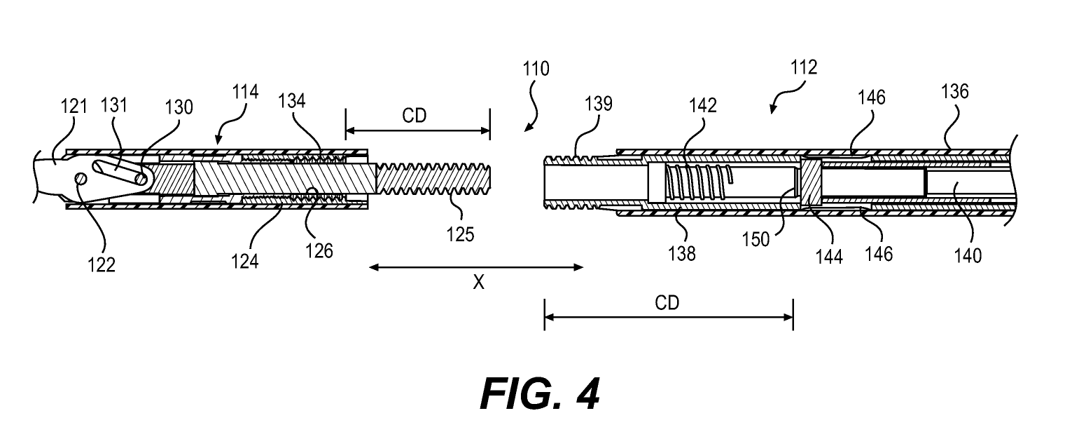

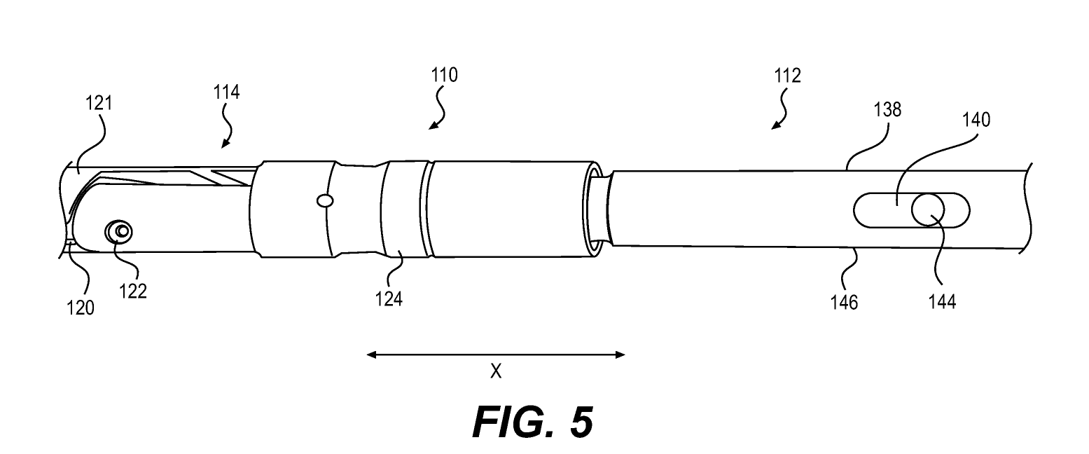

Referring to the drawings wherein like characters represent like elements, FIGS. 4-5 show a laparoscopic instrument assembly 110 having a gaugeless tip arrangement in accordance with the disclosure. At the forward end of the assembly 110, an operable tip 114 is provided with a pair of cutting blades 120, 121 forming a scissors, the blades being movable to an open and closed position by rotation about a pivot pin 122. While the figures show a scissors, it is appreciated by those of skill in the art that other opposable instruments may be employed in accordance with the present invention, including but not limited to graspers, pincers, piercers and the like.

The operable tip 114 includes a tip body, which includes a tubular backhub 124 at the proximal end thereof, and the pair of blades 120, 121 disposed at the distal end thereof. A proximally-extending yoke (or tip rod) 126 is coaxially disposed with the tip body and backhub 124. As shown in FIG. 4, affixed to the yoke 126 is a drive pin 130. Each of the blades 120, 121 has a slotted opening 131 formed at the rear of the pivot pin 122, and the drive pin 130 is received in the slotted openings 131.

The yoke 126 is slidable within the tip body and backhub 124 along X (or longitudinal) axis (shown in FIG. 4), causing the drive pin 130 to slide within the slotted openings 131 to thereby cause the blades 120, 121 to move to an open position with the yoke 126 in a forward (or distal) position and to a closed position with the yoke in its rearwardmost (proximalmost) position. As shown in FIG. 4, the yoke 126 also includes externally-facing screw threads 125, and the backhub 124 includes internally-facing screw threads 134.

Handpiece 112 includes an insulative outer sheath 136 non-rotatably surrounding an outer tube 138 (which may be electrically conductive). The outer tube 138 has an externally-facing thread 139 formed at the distalmost end thereof and having a matching pitch and diameter to the internally-facing threads 134 provided on the backhub 124 such that the backhub and outer tube may be removably and threadedly connectable to each other.

The handpiece 112 also includes an actuator rod (also referred to as an inner shaft) 140 is coaxially disposed within the outer tube 138 and configured to axially slide in the X direction. The inner shaft 140 includes internally-facing threads 142 disposed at the distalmost end thereof. The internally-facing threads 142 are of the same diameter and pitch as externally-facing screw threads 125 of the yoke 126 such that the yoke and inner shaft 140 may be removably and threadedly connectable to each other. By way of the aforementioned yoke-inner shaft threaded engagement and the backhub-outer tube threaded engagement, the tip 114 may be removably and securely attached to the handpiece 112 by what is referred to as a “dual threaded engagement.”

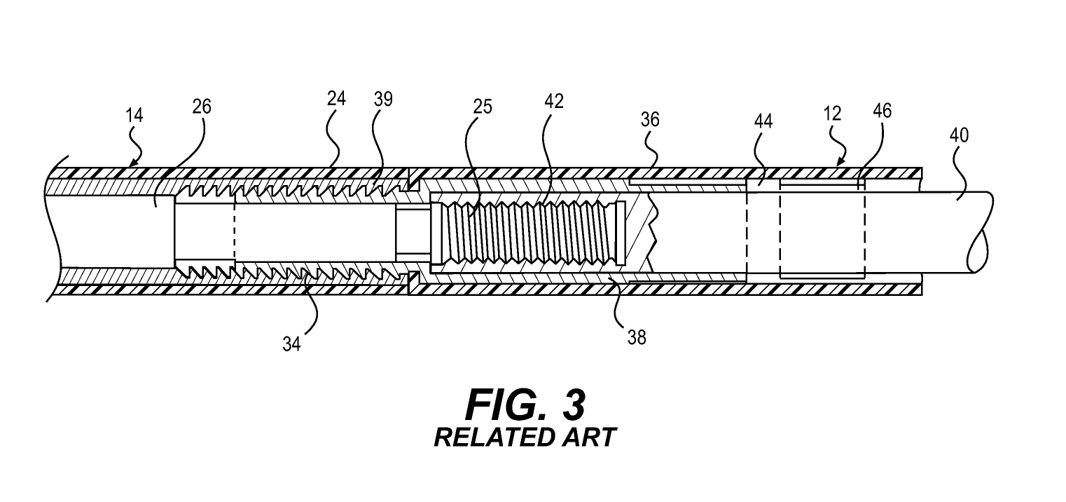

The actuator rod 140 further has a draw pin 144 radially extending therefrom and captured in an elongated slot 146 formed at in the outer tube 138 to prevent rotation of the inner shaft 140 within the outer tube, while providing for movement of the rod in the X direction. The distalmost end of the slot (the left side of FIGS. 3-4) controls the amount of opening of the cutting blades 120, 121, and the proximalmost end of the slot (the right side of FIGS. 3-4) controls the fully closed position of the hand piece before connection with a tip, as well as controls the amount of closure and overstroke of the cutting blades. In other words, when the pin 144 reaches the proximalmost end of the slot the shaft can no longer further close the blades and/or apply pressure to the target site.

More threads per inch (referred to as the thread pitch) are on the outer tube threads 139 and backhub threads 134 than are on the inner shaft threads 142 and yoke threads 125, which means that during the tip 114 attachment process, the inner shaft 140 travels a greater distance in the X direction than the backhub 124 during the tip attachment process (in other words, the inner shaft 140 moves distally in relation to the outer tube 138, which also causes thumb loop 16 to move slightly in the proximal direction). Once the tip 114 and handpiece 112 are completely threaded together such that the backhub 124 abuts against the outer sheath 126, this thread pitch differential resulting in the shaft's longitudinal movement means that the pin 144 is not bottomed out in the proximal end of the slot 146. The resultant space in the slot 146 between the pin 144 and proximal end of the slot 146 allows for overstroke (i.e., allows the user to apply an increased load to the end effector 114 even after the scissors 120, 121 or other opposable implements have touched). It is also noted that the present arrangement also prevents the pin 144 from bottoming out at the distal end of the slot 146, which would otherwise prevent the scissors 120, 121 from fully opening.

In accordance with a feature, the outer tube threads 139 and backhub threads 134 are of a 8-36 thread size, and the yoke threads 125 and inner shaft threads 142 are of a 4-32 thread size, although it is appreciated that different thread sizes may be used in different embodiments. Additionally, the yoke threads 125 and inner shaft threads 142 are threadedly engaged with each other by approximately one winding (one revolution) before the outer tube threads 139 engage with the backhub threads 134.

For a 5 millimeter surgical instrument 110 that uses a coaxial thread design, the precise integration of different components is critical, otherwise damage to the device and/or injury to the patient may result. The proper functioning of the tip 114 is tied directly to the thread pitch and thread differential. The inventors discovered that by utilizing precision components with tight dimensional tolerances in the tip 114, not only could the prior art gauging process be avoided, but hysteresis between the handpiece 112 and the actuation of the end effector could be minimized, thereby improving the user's tactile feel when operating the instrument 110.

For example, a feature of the present disclosure provides a range of dual threaded engagement of 3.971 to 4.390 windings (or revolutions) of the outer tube threads 139 and backhub threads 134. Specifically, once the yoke threads 125 and inner shaft threads 142 have threadedly engaged with each other by approximately one winding, the outer tube threads 139 begin to engage with the backhub threads 134, and the yoke threads 125 and inner shaft threads 142 continue to engage and the outer tube threads 139 and backhub threads 134 continue to engage for 3.971 to 4.390 windings (or revolutions) of the outer tube threads 139 and backhub threads 134. As noted herein “dual threaded engagement” means that both pairs of threads are engaged (i.e., yoke-inner shaft threaded engagement and the backhub-outer tube threaded engagement). Thus, the dual threaded engagement begins when the outer tube threads 139 begin to engage with the backhub threads 134.

It is noted that should the dual threaded engagement be below the range of 3.971 to 4.390 windings, then the scissors 120, 121 (or other opposable elements) may not fully close (bottoming the pin 144 in the proximalmost end of the slot 146), and should the dual threaded engagement be above the range of 3.971 to 4.390 windings, then the scissors (or other opposable elements) may not fully open (bottoming the pin 144 in the distalmost end of the slot 146). Rather, the range of 3.971 to 4.390 windings not only avoids the pin 144 bottoming out in the slot, but provides for proximal movement of the pin 144 within the slot 146 to provide for overstroke, thereby improving the user's tactile feel when operating the instrument 110.

The following is an explanation as to how the inventors obtained the minimum value of 3.971 dual threaded engagement. By analyzing the tolerances of end effectors 114 (including scissor assemblies), a value of 0.002 inches of clearance was measured between the outer diameter of the pivot pin 122 and the hole in which the pivot pin sits; a value of 0.0049 maximum inches of clearance was measured between the drive pin 130 and slotted opening 131; and a value of 0.007 inches of clearance was measured between the pitch diameter difference between both the 4-32 sized yoke threads 125 and inner shaft threads 142. All of these tolerances when added together result in a 0.0139 inch tolerance stackup. Taking into account the dual threading pitch difference of 0.0035 inches (meaning that during the dual threaded engagement, because the yoke 126 and inner shaft 140 travel a greater distance in the X direction per revolution than the backhub 124, for every full revolution of dual threaded engagement, the inner shaft 140 moves 0.0035 inches distally in relation to the outer tube 138, which pulls the pin 144 away from the proximal end of the slot, which ultimately overcomes tolerance stackups and hysteresis and ultimately allows for overstroke), the 0.0139 inch tolerance stackup is divided by the dual threading pitch difference of 0.0035 inches, resulting in a 3.9714 winding dual threaded engagement.

As shown in FIG. 4, an aspect of the disclosure also provides for a constrained dimension CD (defined as the distance between a proximalmost point of the yoke 125 and a beginning of the back hub screw threads 134, with respect to the tip 114; and defined as the distance between the distalmost point of the inner shaft 140 and the proximal end 150 of the receiving space in the inner shaft which accommodates the yoke 126, with respect to the handpiece 112) of between 0.533 inches and 0.608 inches, with an even more preferred range of between 0.533 inches and 0.574 inches. This constrained dimension CD allows the tips 114 of the present disclosure to not only effectively be used with related art handpieces 10. but to also be effectively used with other, specially-designed handpieces 110.

It is noted that should the constrained dimension CD of the tip 114 be less than the range of between 0.533 inches and 0.608 inches, the 3.971 value of dual threaded engagement will not be met, resulting in the risk of the scissors 120, 121 (or other opposable elements) not fully closing. Further, should the constrained dimension CD of the tip 114 be greater than the range of between 0.533 inches and 0.608 inches, the tip 114 will not fully screw onto the handpiece 112 due to the yoke 126 bottoming out in proximal end 150 of the receiving space in the inner shaft 140 (since an aspect of the disclosure provides a minimum thread depth of 0.200 inches), thereby creating a gap between the backhub 124 and outer sheath 136, and possibly creating electrical and/or fluid leakage of the instrument 110.

A method of attaching the tip 114 to the handpiece 112 will now be described. With the jaws 120, 121 of the tip 114 closed and the thumb and finger grip members 16 and 17 closed, and without rotating the outer tube 138 relative to the inner shaft 140, the tip is twisted by the user relative to the handpiece such that the yoke screw threads 125 threadedly engage with the shaft screw threads 142. Thereafter, while still twisting the tip, the back hub screw threads 134 threadedly engage with the outer tube threads 139, whereupon that the inner shaft distally moves in relation to the outer tube. Installation is complete when the user can no longer twist the tip 114 relative to the handpiece 112.

In an aspect of the disclosure, handpiece hysteresis is minimized by the use of a ball and socket connection between the inner shaft 140 and thumb loop 16 to provide smooth and precise movement of the inner shaft within the outer tube 138. Specifically, the ball and socket connection maintains the angular relationship of the inner shaft 140 to the outer tube 138 when rotated within the handpiece 112 (i.e. , no matter where the inner shaft 140 and outer tube 138 are positioned rotationally to the handle, the angular relationship between the ball of the inner shaft and the socket of the hand piece are maintained). To the contrary, when using a clevis instead of a socket, the inherent design constraints of the clevis means that this angular relationship changes as the inner shaft 140 and the outer tube 138 are rotated with respect to the handpiece 112. This means that with a clevis design, the amount of “opening” or “closing” of a tip 114 that happens when the handles 16, 17 are opened or closed a specific amount changes depending on the rotational alignment of the inner shaft 140 and outer tube 138 to the handles 16, 17. Thus, a ball and socket arrangement, when used in conjunction with the instrument 110, is much tighter than a ball and clevis arrangement, so it does not significantly contribute to hysteresis in the system.

In view of the foregoing, the present disclosure, through one or more of its various aspects, embodiments and/or specific features or sub-components, is thus intended to bring out one or more of the advantages as specifically noted below. While the present disclosure includes description with respect to a medical procedure, the present invention may he used in a variety of other, non-medical, environments.

The illustrations of the embodiments described herein are intended to provide a general understanding of the structure of the various embodiments. The illustrations are not intended to serve as a complete description of all of the elements and features of apparatus and systems that utilize the structures or methods described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein. it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. §1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

What is claimed is:

1. A removable tip attachable to an instrument handpiece, the tip comprising:
a tip body;
a back hub at a proximal end of the tip body and having internally-facing back hub screw threads configured to threadedly engage outer externally-facing tube threads of an outer tube of the instrument handpiece;
an end effector at a distal end of the tip body;
a yoke at the proximal end of the tip body and having externally-facing yoke screw threads configured to threadedly engage internally-facing shaft screw threads of an inner shaft of the instrument handpiece, the yoke configured to slidably and coaxially move within the backhub to actuate the end effector upon sliding actuation of the inner shaft within the outer tube, wherein:
the yoke screw threads engage with the inner shaft screw threads before the back hub screw threads engage the outer tube threads upon attachment of the tip to the instrument handpiece; and
the distance between a proximalmost point of the yoke and a beginning of the back hub screw threads is between 0.533 inches and 0.608 inches.

2. The removable tip according to claim 1, wherein the distance between a proximalmost point of the yoke and a beginning of the back hub screw threads is between 0.533 inches and 0.574 inches.

3. The removable tip according to claim 1, wherein the back hub screw threads are configured to engage the outer tube threads for 3.971 to 4.390 windings after the yoke screw threads engage with the inner shaft screw threads.

4. The removable tip according to claim 3, wherein the back hub screw threads are configured to engage the outer tube threads for 3.971 windings after the yoke screw threads engage with the inner shaft screw threads.

5. The removable tip according to claim 1, wherein the backhub has an outer diameter of 5 millimeters.

6. The removable tip according to claim 1, wherein:
the back hub screw threads are a 8-36 screw size; and
the yoke screw threads are a 4-32 screw size.

7. A method of removably attaching an instrument tip to an instrument handpiece, the instrument tip having:
a back hub at a proximal end of a tip body and having internally-facing back hub screw threads,
an actuable end effector at a distal end of the tip body; and
a yoke coaxially disposed within the tip body, extending from the proximal end of the tip body and having externally-facing yoke screw threads; and
the instrument handpiece having:
an outer tube having externally-facing tube threads; and
an inner shaft slidably disposed within the outer tube and having internally-facing shaft screw threads;
the method comprising:
threadedly engaging the yoke screw threads with the shaft screw threads by at least one winding; and
thereafter, threadedly engaging the back hub screw threads with the tube threads within a range of 3.971 and 4.390 windings such that the inner shaft distally moves in relation to the outer tube.

8. The method according to claim 7, wherein during the threadedly engaging the back hub screw threads with the tube threads, the end effector is not actuated.

9. The method according to claim 7, wherein:
the inner shaft includes a pin radially extending therefrom, and slidable within an axially extending slot located in the outer tube, the method further comprising:
during the threadedly engaging the back hub screw threads with the tube threads, the pin remains out of contact with opposed ends of the slot.

10. The method according to claim 7, wherein the distance between a proximalmost point of the yoke and a beginning of the back hub screw threads is between 0.533 inches and 0.608 inches.

11. The method according to claim 7, wherein the distance between a proximalmost point of the yoke and a beginning of the back hub screw threads is between 0.533 inches and 0.574 inches.

12. The method according to claim 7, wherein the threadedly engaging the back hub screw threads with the tube threads is 3.971 windings.

13. The method according to claim 7, wherein:
the back hub screw threads are a 8-36 screw size; and
the yoke screw threads are a 4-32 screw size.

14. An instrument assembly comprising:
an instrument tip comprising:
a back hub at a proximal end of a tip body and having internally-facing back hub screw threads,
an actuable end effector at a distal end of the tip body; and
a yoke coaxially disposed within the tip body, extending from the proximal end of the tip body and having externally-facing yoke screw threads; and
an instrument handpiece for removably accepting attachment of the tip thereto, comprising:
an inner shaft slidably disposed within the outer tube and having internally-facing shaft screw threads threadedly engaging the yoke screw threads;
an outer tube having externally-facing tube threads threadedly engaging the back hub screw threads for 3.971 to 4.390 windings after the shaft screw threads engage with the yoke screw threads, such that the tip is affixed to the handpiece.

15. The assembly according to claim 14, wherein the tube threads engage the back hub screw threads for 3.971 windings after the shaft screw threads engage with the yoke screw threads.

16. The assembly according to claim 14, further comprising:
a pin radially extending from the inner shaft; and
an axially extending slot located in the outer tube and capturing the pin therewithin, such that the pin remains out of contact with opposed ends of the slot in an unactuated state of the tip.

17. The assembly according to claim 14, wherein the distance between a proximalmost point of the yoke and a beginning of the back hub screw threads is between 0.533 inches and 0.608 inches.

18. The assembly according to claim 14, wherein the distance between a proximalmost point of the yoke and a beginning of the back hub screw threads is between 0.533 inches and 0.574 inches.

19. The assembly according to claim 14, further comprising an outer sheath covering the outer tube, wherein the backhub and the outer sheath each have an outer diameter of 5 millimeters.

20. The assembly according to claim 14, wherein:
the back hub screw threads and outer tube screw threads are each a 8-36 screw size; and
the yoke screw threads and inner shaft screw are each a 4-32 screw size.
